(19) United States
(12) Patent Application Publication
(10) Pub. No.: US 2025/0242117 A1
(43) Pub. Date: Jul. 31, 2025

(54) POWDER INHALER

(71) Applicant: APTAR FRANCE SAS, Le Neubourg (FR)

(72) Inventor: Matthieu BAILLET, Rouen (FR)

(73) Assignee: APTAR FRANCE SAS, Le Neubourg (FR)

(21) Appl. No.: 18/855,056

(22) PCT Filed: Apr. 13, 2023
(86) PCT No.: PCT/FR2023/050531

§ 371 Date: Oct. 8, 2024

(30) Foreign Application Priority Data
Apr. 14, 2022 (FR) FR2203475

(51) Int. Cl. A61M 15/00; A61M 15/08

(52) U.S. Cl. CPC A61M 15/0041; A61M 15/08; A61M 2202/064

(57) ABSTRACT

The invention relates to a powder inhaler comprising a body (10) for receiving a reservoir (20) having a cavity (21) containing a dose of powder and a closing layer (22) for sealingly closing the cavity (21), the body (10) having a head portion (11) and two support portions (12a, 12b), the head portion (11) being connected to each support portion (12a, 12b) by at least one joint (13a, 13b), the head portion (11) having a dispensing opening (110) and a perforating element (112) connected to the dispensing opening (110), which perforating element is designed, when actuated, to pierce the closing layer (22) in order to open the reservoir (20) arranged on a support portion (12a, 12b), a first support portion (12a) for receiving a first reservoir containing a first dose and a second support portion (12b) for receiving a second reservoir containing a second dose, the body (10) being formed of a single piece.

The present invention relates to a powder inhaler.

Powder inhalers, in particular of the pharmaceutical type, are well known in the prior art. Various types exist. A first type of inhaler contains a reservoir receiving many doses of powder, the inhaler being provided with metering means making it possible, on each actuation, to remove one dose of said powder from the reservoir, so as to bring said dose into an expulsion duct in order to be dispensed to the user. Another type of inhaler consists in placing the doses of powder in individual predosed reservoirs, then in opening one of the reservoirs each time the inhaler is actuated. That implementation seals the powder more effectively since each dose is opened only when it is about to be expelled. In order to make such individual reservoirs, various techniques have been proposed, such as an elongate blister strip or blisters disposed on a rotary circular disk. All existing types of inhalers, including those described above, present both advantages and drawbacks associated with their structures and with their types of operation. Multidose inhalers and inhalers containing a plurality of individual reservoirs are generally complex devices, constituted by a large number of parts, and thus costly to manufacture and to assemble.

In order to make devices less complex and thus less costly, unidose inhalers have been proposed that include one single individual reservoir, such as a capsule or a blister, which is to be loaded into the inhaler before it is used. The advantage of such devices is that it is not necessary to store all of the doses inside the appliance, such that said appliance can be compact.

Documents WO2007129127, WO2007129128 and WO2012004485 describe prior art devices. These devices have drawbacks, for example a difficult assembly of the individual reservoir in a unidose inhaler, which makes it complicated to use, for example in the event of a crisis or for people with disabilities. Likewise, these devices generally have several parts, which complicates manufacture and assembly.

Documents WO2016091386 and WO2007144614 describe other devices of the prior art. Document WO2016091386 describes a clothes peg-shaped unidose device, and document WO2007144614 describes an oral inhaler.

None of these documents describes a bidose device adapted to dispense two doses successively, for example a first dose in a first nostril and a second dose in a second nostril.

An object of the present invention is to provide a powder inhaler that does not have the above-mentioned drawbacks.

In particular, an object of the present invention is to provide such an inhaler that is simple and inexpensive to manufacture and to assemble, and that is reliable in use.

The object of the present invention therefore is a powder inhaler, comprising a body for receiving a reservoir having a cavity containing a dose of powder and a closing layer for sealingly closing said cavity, said body having a head portion and two support portions, said head portion being connected to each support portion by at least one joint, said head portion having a dispensing opening and a perforating element connected to said dispensing opening, which perforating element is designed, when actuated, to pierce said closing layer in order to open said reservoir arranged on a support portion, a first support portion for receiving a first reservoir containing a first dose and a second support portion for receiving a second reservoir containing a second dose, said body being formed of a single piece.

Advantageously, said dispensing opening is formed at the axial end of a nasal endpiece intended to be inserted into a nostril during actuation.

Advantageously, each joint is formed by a bridge of foldable material.

Advantageously, said head portion has at least one window and each support portion has at least one projection for engaging in the closed position of the inhaler with a respective window, in particular by snap-fitting, to lock said closed position.

Advantageously, said locking of the closed position is unlockable, to enable the body to be reused with several reservoirs.

Advantageously, each support portion has a recess adapted to house said cavity of said reservoir.

Advantageously, said reservoir has at least one positioning opening, each support portion having at least one stud for receiving a respective positioning opening.

Advantageously, after positioning said reservoir on a support portion, each stud is deformed, in particular snapped, in order to non-removably fix said reservoir on said support portion.

Advantageously, said head portion has at least one blocking tab for blocking the inhaler in an intermediate position in which said perforating element has not pierced said closing layer of said reservoir, such that during actuation, the user must break or deform said at least one blocking tab in order to be able to actuate the inhaler.

Advantageously, said two support portions are arranged symmetrically on either side of said head portion.

These features and advantages and others of the present invention will appear more clearly during the following detailed description, made in reference to the accompanying drawings, given as non-limiting examples, and in which

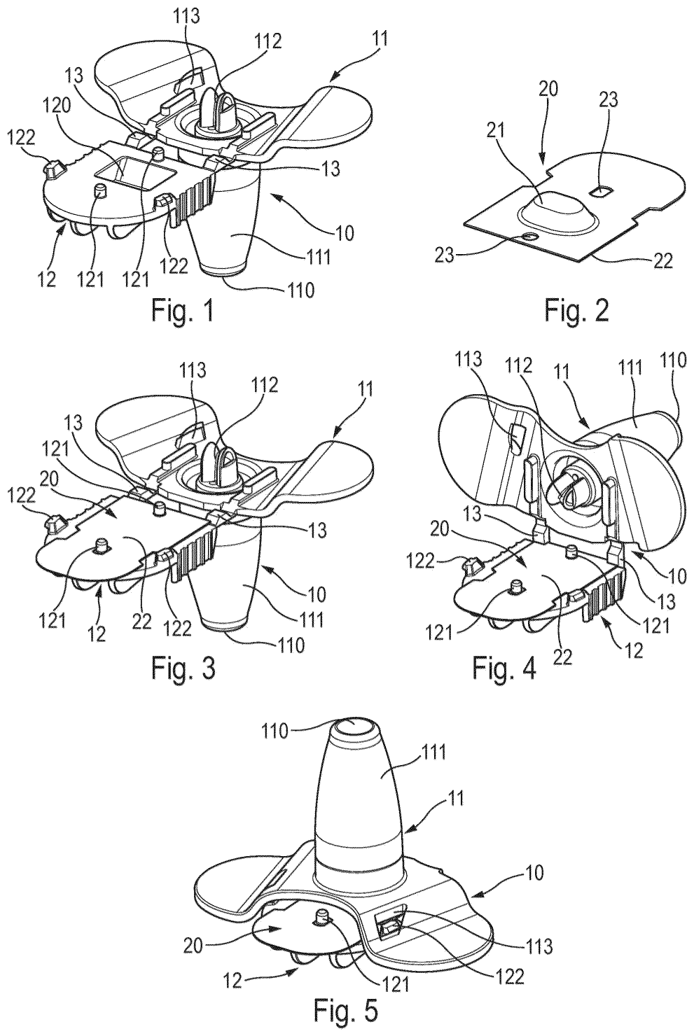

FIG. 1 is a perspective, schematic view of a unidose powder inhaler according to a first advantageous embodiment, before assembly of the reservoir and in the open position of the inhaler,

FIG. 2 is a perspective, schematic view of a reservoir which can be adapted to the inhaler of FIG. 1,

FIG. 3 is a perspective, schematic view similar to that of FIG. 1, with the reservoir assembled in the inhaler, in the open position of the inhaler,

FIG. 4 is a view similar to that of FIG. 3, during the opening of the inhaler,

FIG. 5 is a view similar to that of FIG. 4, in the closed position of the inhaler,

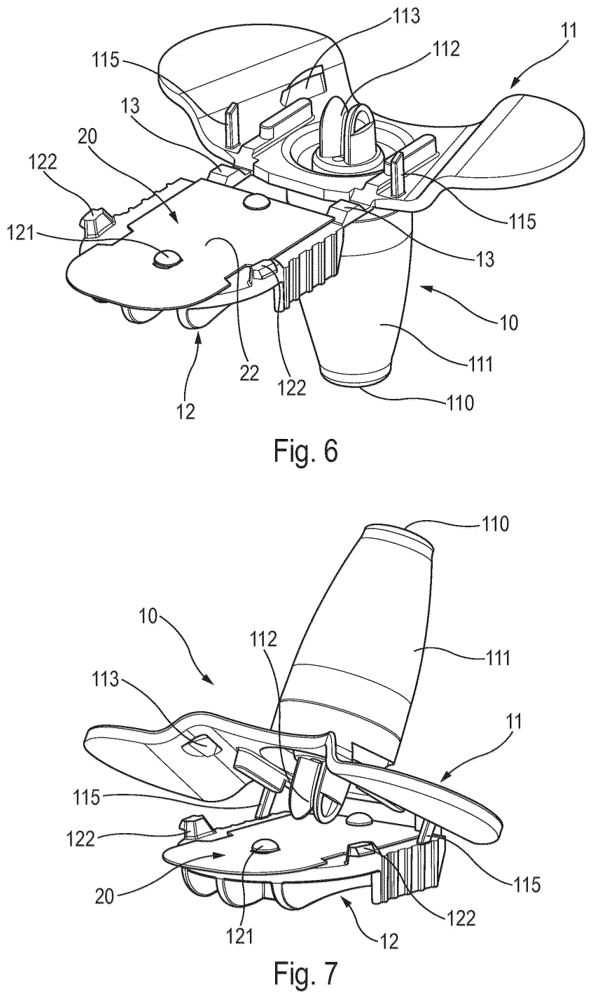

FIG. 6 is a view similar to that of FIG. 3, showing a unidose powder inhaler according to another advantageous embodiment,

FIG. 7 is a view similar to that of FIG. 6, in the intermediate position of the inhaler,

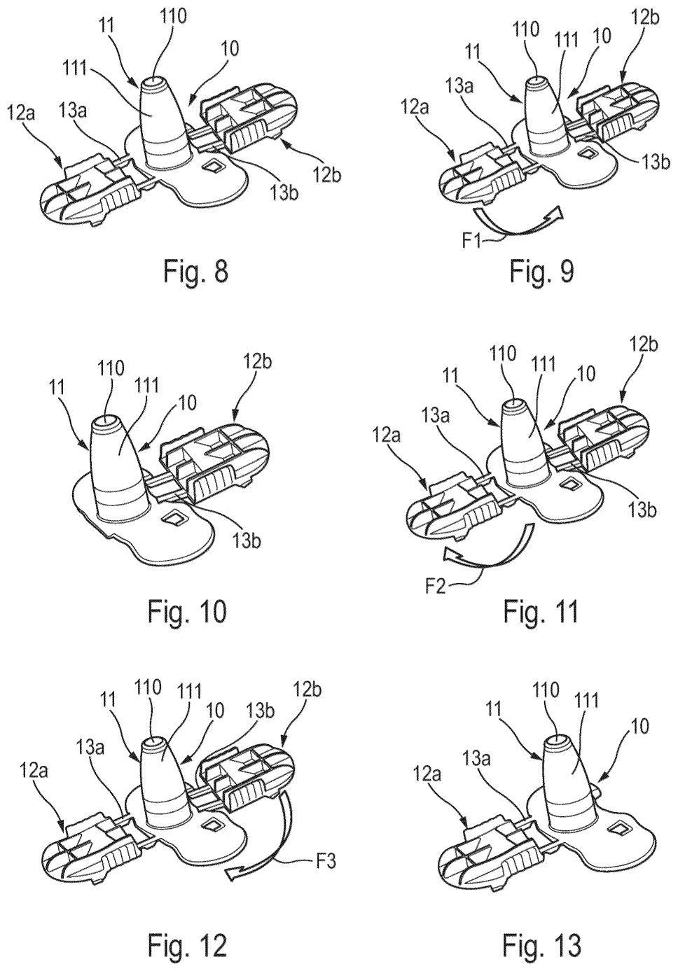

FIG. 8 is a very schematic view of a bidose powder inhaler according to an advantageous embodiment of the invention, in the open position of the inhaler before its first use,

FIG. 9 is a view similar to that of FIG. 8, showing the pivoting of the first portion of the support to dispense a first dose,

FIG. 10 is a view similar to that of FIG. 9, showing the device ready for the dispensing of the first dose,

FIG. 11 is a view similar to that of FIG. 10, showing the pivoting of the first support portion after dispensing of the first dose,

FIG. 12 is a view similar to that of FIG. 11, showing the pivoting of the first support portion to dispense a first dose,

FIG. 13 is a view similar to that of FIG. 12, showing the device ready for the dispensing of the second dose.

FIGS. 1 to 7 describe first advantageous embodiment, relating to a unidose device. This first embodiment describes in detail, all the features which apply to the embodiment according to the invention of FIGS. 8 to 13.

The inhaler has a single-piece body 10 formed of one single piece by moulding a synthetic material. The body 10 has a head portion 11 and a support portion 12, said portions 11, 12 being connected by at least one joint 13. Thus, the body 10 is deformable between an open position, shown in FIGS. 1 and 3, and a closed position, shown in FIG. 5. Preferably, the head 11 and support 12 portions pivot with respect to the joint 13 by around 180° between the open and closed positions. Advantageously, the joint(s) 13 is/are formed by one or more bridge(s) of foldable material.

The head portion 11 has a dispensing opening 110, preferably formed at the axial end of a nasal endpiece 111 that the user places in their nostril when using the inhaler. The dispensing opening 110 is connected to a perforating element 112. Advantageously, the head portion further has at least one window 113, advantageously two.

The support portion 12 is intended to receive an individual reservoir 20, for example, shaped as a blister. An example of such a reservoir 20 is shown in FIG. 2. This reservoir 20 has a cavity 21 containing a dose of powder, and a closing layer 22 to sealingly close said cavity 21. The closing layer 22 is advantageously made of a material which is adapted to protect the powder contained in the cavity 21, in particular against humidity, for example, a metal such as aluminium or a suitable multilayer structure. The reservoir 20 has at least one positioning opening 23, preferably two.

The support portion 12 has a recess 120 adapted to house the cavity 21 of the reservoir 20. It also has at least one stud 121, preferably two, to receive the positioning openings 23 of the reservoir 20. It also has at least one projection 122, preferably two, for engaging in the closed position of the inhaler with the windows 113 of the head portion 11, in particular by snap-fitting, to lock said closed position. Advantageously, each projection 122 has a snap-fitting profile which will snap-fit into a respective window 113 in the closed position, as can be seen in FIG. 5.

Advantageously, this locking can be unlocked, which makes it possible to reopen the inhaler to replace the empty reservoir with a new full reservoir, and thus to reuse the inhaler to dispense several doses. In this case, the projections 122 can have a different shape, with the snap-fitting profile rotated inwards, rather than outwards as in the examples of FIGS. 1 to 7.

When the user wishes to use the inhaler, they take the body 10 in the open position and position a reservoir 20 on the support portion 12. The cavity 21 of the reservoir 20 is arranged in the recess 120 and the positioning openings 23, two of them in the example shown, are arranged around the studs 121, also two of them. The closing layer 22 is therefore rotated upwards, in the position such as shown in FIG. 3.

The user thus closes the body 10 by pivoting the head portion 11 around 180° on the support portion 12 around the joints 13, which are two in the example shown. During this closing movement, the perforating element 112 will pierce the closing layer 22 of the reservoir 20 and penetrate into the cavity 21. In the closed position, the projections 122 of the support portion 12 are snap-fitted into the windows 113 of the head portion 11 to lock this closed position during actuation.

The user thus places the nasal endpiece 111 in a nostril and suctions, which dispenses the powder contained in the cavity 21 through the perforating element 112 and the dispensing opening 110.

If the inhaler is single-use, it is disposed of. In this case, the locking of the closed position is preferably definitive. If, on the contrary, the inhaler can be reused, the user unlocks the closed position and returns the body 10 into the open position, in which the empty reservoir can be removed from the support portion 12. Advantageously, the body 10, in particular the head portion 11, can be cleaned or washed before another use.

In the variant of the embodiment of FIGS. 6 and 7, the reservoir 20 can be non-removably fixed to the support portion 12. For example, after having arranged the reservoir 20 on the support portion 12, with the cavity 21 arranged in the recess 120 and the positioning openings 23 arranged around the studs 121, the latter can be deformed, for example, by snapping. In this way, the studs 121 widen and are blocking the reservoir 20 on the support portion. In a variant, a snap-fitting of the positioning openings 23 on the studs 121 could be considered, which could thus have an adapted profile.

This implementation makes it possible to pre-install the reservoir 20 in the inhaler, and to deliver the assembly thus pre-assembled. The user thus no longer needs to position a reservoir on the support portion 12 before using the inhaler, but only to close the latter.

The head portion 11 in the example of FIGS. 6 and 7 can also have at least one blocking tab 115, preferably two, for blocking the inhaler in an intermediate position in which the perforating element 112 has not pierced the closing layer 22 of the reservoir 20. This intermediate position can be seen in FIG. 7. In this intermediate position, the end of each blocking tab 115 abuts on a surface of said support portion 12. During actuation, the user must thus break or deform these blocking tabs 115 in order to be able to actuate the inhaler. In this example, the device can be delivered in the intermediate position, with the reservoir 20 pre-installed, and the user, to actuate, thus only needs to press hard enough on the body 10 to break or deform the blocking tabs 115 and thus bring the body 10 into the closed position, with the perforating element 112 which has pierced the closing layer 22 of the reservoir to enable the inhalation of the dose of powder.

FIGS. 8 to 13 very schematically describe an advantageous embodiment of the invention, relating to a bidose device. This embodiment corresponds to a bidose version of the device described in the first embodiment, and all the features and variants described in the scope of this first embodiment in reference to FIGS. 1 to 7 apply to this embodiment.

According to the invention, the body 10 has two support portions 12a, 12b, each being connected to the head portion 11 by at least one joint 13a, 13b. Preferably, these two support portions 12a, 12b are arranged symmetrically on either side of the head portion, and they are advantageously identical.

The first support portion 12a receives a first reservoir containing a first dose and the second support portion 12b receives a second reservoir containing a second dose.

To dispense the first dose, the user pivots the first support portion 12a having its respective reservoir by around 180°, as illustrated by the arrow F1 in FIG. 9, to dispense the first dose.

After dispensing of the first dose, it returns the first support portion 12a to its initial position, as illustrated by the arrow F2 in FIG. 11, then pivots the second support portion 12b having its respective reservoir by around 180°, as illustrated by the arrow F3 in FIG. 12, to dispense the second dose.

Various modifications are possible for a person skilled in the art without departing from the scope of the present invention such as defined by the accompanying claims.

1. A powder inhaler, comprising a body for receiving a reservoir having a cavity containing a dose of powder and a closing layer to sealingly close said cavity, characterised in that said body has a head portion and two support portions, said head portion being connected to each support portion by at least one joint, said head portion having a dispensing opening and a perforating element connected to said dispensing opening, which perforating element is designed, when actuated, to pierce said closing layer to open said reservoir arranged on a support portion, a first support portion for receiving a first reservoir containing a first dose and a second support portion for receiving a second reservoir containing a second dose, said body being formed of a single piece.

2. The powder inhaler according to claim 1, wherein said dispensing opening is formed at the axial end of a nasal endpiece intended to be inserted into a nostril during actuation.

3. The powder inhaler according to claim 1, wherein each joint is formed by a bridge of foldable material.

4. The powder inhaler according to claim 1, wherein said head portion has at least one window and each support portion has at least one projection for engaging in the closed position of the inhaler with a respective window, in particular by snap-fitting, to lock said closed position.

5. The powder inhaler according to claim 4, wherein said locking of the closed position is unlockable, to enable the body to be reused with several reservoirs.

6. The powder inhaler according to claim 1, wherein each support portion has a recess adapted to house said cavity of said reservoir.

7. The powder inhaler according to claim 1, wherein said reservoir has at least one positioning opening, each support portion having at least one stud for receiving a respective positioning opening.

8. The powder inhaler according to claim 7, wherein after positioning said reservoir on a support portion, each stud is deformed, in particular snapped, to non-removably fix said reservoir on said support portion.

9. The powder inhaler according to claim 1, wherein said head portion has at least one blocking tab for locking the inhaler in an intermediate position in which said perforating element has not pierced said closing layer of said reservoir, such that during actuation, the user must break or deform said at least one blocking tab in order to be able to actuate the inhaler.

10. The powder inhaler according to claim 1, wherein said two support portions are arranged symmetrically on either side of said head portion.
